(19) United States
(12) Patent Application Publication
(10) Pub. No.: US 2026/0232912 A1
(43) Pub. Date: Aug. 13, 2026

(54) SYRINGE ASPIRATION SAFETY ASSISTANCE DEVICE

(71) Applicant: YU-TING TSAI, Taoyuan City (TW)

(72) Inventor: YU-TING TSAI, Taoyuan City (TW)

(21) Appl. No.: 19/424,910

(22) Filed: Dec. 18, 2025

(30) Foreign Application Priority Data
Feb. 12, 2025 (TW) 114201467

(51) Int. Cl. A61M 5/31; A61M 5/315

(52) U.S. Cl. CPC A61M 5/3148; A61M 5/31501; A61M 2205/586

(57) ABSTRACT

A syringe aspiration safety assistance device includes a reverse-buckle finger sleeve, a syringe stop plate, and a syringe groove plate. The reverse-buckle finger sleeve has an upper end that extends to form a hook-shaped structure, and a lower end connected to one end of the syringe stop plate. The syringe stop plate has a lower end connected to an inner side of the syringe groove plate. The syringe groove plate has a syringe groove.

### FIELD OF THE DISCLOSURE

The present disclosure relates to a syringe aspiration safety assistance device, and more particularly to a device that enables aspiration to be performed using only a single-joint motion of the thumb of an injecting hand.

### BACKGROUND OF THE DISCLOSURE

In recent years, medical aesthetic procedures have become increasingly popular, especially facial filler treatments used by individuals of varying ages and genders. However, the injection of fillers carries certain risks. If a micro-aesthetic filler (such as hyaluronic acid, collagen, collagen stimulators, and the like) is inadvertently injected into a blood vessel, embolism may occur, potentially causing severe and life-threatening conditions including localized tissue necrosis, stroke, pulmonary embolism, and blindness. Accordingly, an aspiration technique is typically employed during filler injections to eliminate the possibility that the needle tip is located in a blood vessel.

In conventional original syringes designed by major filler manufacturers currently on the market, when an injector performs aspiration, a change in hand posture is generally required, and the aspiration duration must be maintained for at least more than five seconds. This results in difficulty in the injector's controlling the stability of the syringe needle tip under the skin, and causes deviation of the injection position, thereby leading to injection of the filler into a blood vessel.

In addition, conventional aspiration devices and techniques also have multiple problems, resulting in many stability issues and difficulty in control during aspiration.

For instance, U.S. Pat. No. 11,173,248 B2 discloses a device with an opening for insertion of the upper end of a syringe plunger. However, because the syringe plunger is not clamped and fixed in place, the operator needs to press and hold the upper end of the syringe plunger with the thumb in order to perform aspiration. As a result, the device may detach from the syringe plunger during aspiration, and the ring-shaped configuration of the device restricts the range of movement of the thumb joint, resulting in unsmooth movement and difficult control during pressing and aspirating operations of the syringe.

U.S. Pat. No. 11,464,910 B1 discloses a novel syringe, but the problem therein is that it cannot be used with the conventional original syringe structures supplied by every major filler manufacturer.

U.S. Pat. No. 10,980,945 B1 discloses a device formed from an elastic band made of a flexible material. Devices of this type are generally intended for single use and are not reusable. Furthermore, they suffer from the same compatibility limitations as those described in U.S. Pat. No. 11,464,910B1 .

U.S. Patent Application No. 2015/0238698 A1 describes an invention similar to that of U.S. Pat. No. 11,173,248B2 , with the only difference lying in the design of the reverse-buckle ring. It has the same problem as that in U.S. Pat. No. 11,464,910B1.

Finally, WIPO Patent Application No. WO 2022/008202 A1 describes a two-piece combined device in which a thumb ring must match the shape of the upper end of the syringe plunger in order to be combined. Consequently, the number of conventional original syringe designs supplied by major filler manufacturers, available on the market, and compatible with the device is very limited.

In summary of the above prior art and their problems, it can be understood that there is an urgent market demand for a device that can be adapted to the conventional original syringe designs of major filler manufacturers currently on the market and can solve the problem of deviation of the injection position.

### BRIEF SUMMARY OF THE DISCLOSURE

In view of the aforementioned drawbacks of the prior art, it is an objective of the disclosure to provide a syringe aspiration safety assistance device. The device comprises a reverse-buckle finger sleeve, a syringe stop plate, and a syringe groove plate. An injector performs aspiration by securing an upper end of a syringe plunger between the syringe stop plate and a syringe groove of the syringe groove plate, while inserting a thumb of an injecting hand into the reverse-buckle finger sleeve through a hook-shaped structure formed by extending an upper end of the reverse-buckle finger sleeve, thereby permitting aspiration through a single-joint motion of the thumb in a standard injection posture.

Another objective of the disclosure is to provide a syringe aspiration safety assistance device conducive to reduction of posture changes during aspiration, thereby improving the stability of the injector and improving the accuracy and safety of injection through good stability.

Yet another objective of the disclosure is to provide a syringe aspiration safety assistance device adaptable to the vast majority of brand-name, original filler syringes available on the market.

The problem to be solved by the disclosure is that in the original syringe designs of major filler manufacturers currently on the market, when the injector performs aspiration, a change in hand posture is generally required, and the aspiration duration must be maintained for at least more than five seconds, resulting in difficulty in the injector's controlling the stability of the syringe needle tip under the skin, which easily causes deviation of the injection position and consequently leads to injection of the filler into a blood vessel, thereby raising doubt as to whether its safety is sufficient. Therefore, there is an urgent need for improvement of the accuracy and safety of aspiration.

To achieve the above and other objectives, the disclosure provides a syringe aspiration safety assistance device, comprising:

a syringe groove plate having a syringe groove;

a syringe stop plate having a lower end connected to an inner side of the syringe groove plate; and

a reverse-buckle finger sleeve having an upper end extending to form a hook-shaped structure, and a lower end connected to an end of the syringe stop plate.

The syringe aspiration safety assistance device has a weight of 8 grams.

The syringe aspiration safety assistance device is made of a material including, but not limited to, metal, plastic, or combinations thereof.

The syringe stop plate and the syringe groove plate forcibly clamp an upper end of a plunger of a syringe through elasticity of the material of the syringe stop plate and the syringe groove plate.

The syringe stop plate and the syringe groove plate undergo elastic deformation to receive the upper end of the plunger of the syringe when the upper end of the plunger of the syringe has variable thickness.

The syringe aspiration safety assistance device is made of an aluminum alloy.

The syringe stop plate and the syringe groove plate undergo elastic deformation to receive the upper end of the plunger of the syringe when the upper end of the plunger of the syringe has variable thickness.

Edges of the syringe aspiration safety assistance device are subjected to rounded chamfer processing.

The reverse-buckle finger sleeve has a one-way opening.

The one-way opening and the hook-shaped structure are configured to allow movement of a thumb.

One end of the hook-shaped structure of the reverse-buckle finger sleeve is a trapezoidal structure. reverse-buckle finger sleeve.

The trapezoidal structure is configured to enhance convenience and mobility of a thumb entering and exiting the reverse-buckle finger sleeve.

The length of the reverse-buckle finger sleeve is 22 mm, and the outer radius of the hook-shaped structure is 9.5 mm.

The length of the reverse-buckle finger sleeve is 24 mm, and an outer radius of the hook-shaped structure is 10.5 mm.

The syringe groove of the syringe groove plate has a shape including, but not limited to, a U-shape.

An inner side of an upper end of the syringe groove plate has a step.

The shape of the syringe groove ensures that a syringe clamped by the syringe stop plate and the syringe groove plate does not slide during use.

The distance between the syringe stop plate and the syringe groove plate is 3 mm.

The aspiration is performed through a single-joint motion of a thumb in a standard injection posture.

Compared with the benefits of the prior art, the disclosure provides a syringe aspiration safety assistance device comprising a reverse-buckle finger sleeve, a syringe stop plate, and a syringe groove plate. The reverse-buckle finger sleeve has an upper end that extends to form a hook-shaped structure, and a lower end connected to one end of the syringe stop plate. The syringe stop plate has a lower end connected to an inner side of the syringe groove plate. The syringe groove plate has a syringe groove. After the upper end of the plunger of the syringe has been secured between the syringe stop plate and the syringe groove of the syringe groove plate, a thumb of an injecting hand of an injector is inserted into the reverse-buckle finger sleeve through the hook-shaped structure formed by extending the upper end of the reverse-buckle finger sleeve, thereby permitting aspiration through a single-joint motion of the thumb of the injecting hand of the injector in a standard injection posture, thereby improving stability, accuracy, and safety during aspiration.

### BRIEF DESCRIPTION OF THE DRAWINGS

It should be noted that the drawing figures may be in simplified form and might not be of a precise scale. In reference to the disclosure herein, for purposes of convenience and clarity only, directional terms such as top, bottom, left, right, up, down, over, above, below, beneath, rear, front, distal, and proximal are used with respect to the accompanying drawings. Such directional terms should not be construed to limit the scope of the embodiment in any manner.

FIG. 1 is a perspective view of a syringe aspiration safety assistance device of the disclosure.

FIG. 2 is a side view of the syringe aspiration safety assistance device of the disclosure.

FIG. 3 is a top view of the syringe aspiration safety assistance device of the disclosure.

FIG. 4 is a perspective view showing the syringe aspiration safety assistance device fixing a syringe in place according to the disclosure.

### DETAILED DESCRIPTION OF THE EMBODIMENTS

The different aspects of the various embodiments can now be better understood by turning to the following detailed description of the embodiments, which are presented as illustrated examples of the embodiments defined in the claims. It is expressly understood that the embodiments as defined by the claims may be broader than the illustrated embodiments described below.

The term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

Unless defined otherwise, all technical and position terms used herein have the same meaning as commonly understood by one of ordinary skill in the art to which the invention pertains. Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

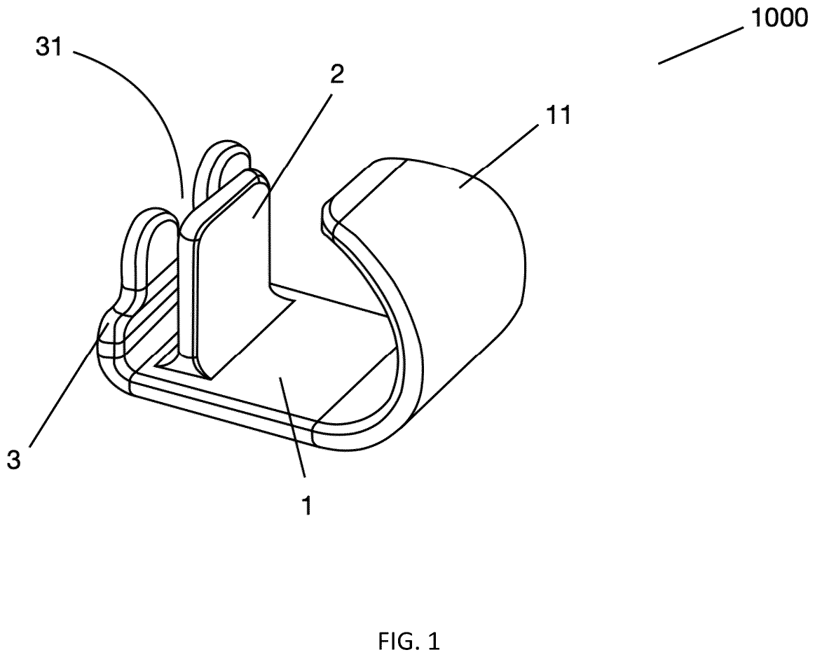

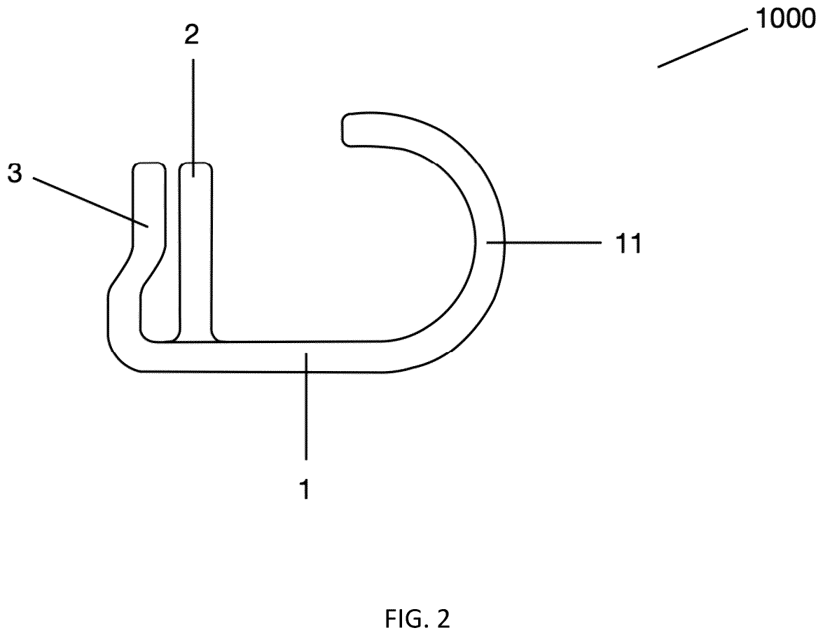

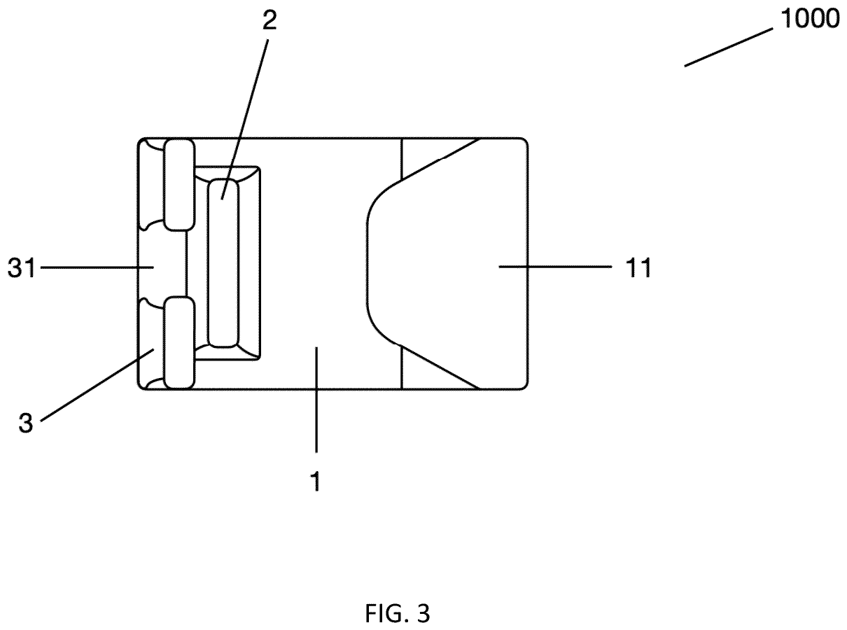

Referring to FIG. 1, FIG. 2, and FIG. 3, which are the perspective view, side view, and top view of the present disclosure, a syringe aspiration safety assistance device 1000 of the disclosure comprises a reverse-buckle finger sleeve 1, a syringe stop plate 2, and a syringe groove plate 3. The upper end of the reverse-buckle finger sleeve 1 extends to form a hook-shaped structure 11, and the lower end is connected to one end of the syringe stop plate 2. The lower end of the syringe stop plate 2 is connected to an inner side of the syringe groove plate 3. The syringe groove plate 3 has a syringe groove 31. In a preferred embodiment of the disclosure, the syringe aspiration safety assistance device 1000 comprises a reverse-buckle finger sleeve 1, a syringe stop plate 2, and a syringe groove plate 3.

The syringe aspiration safety assistance device 1000 is made of a material including, but not limited to, metal, plastic, or combinations thereof.

The syringe aspiration safety assistance device 1000 is made of an aluminum alloy.

When the syringe aspiration safety assistance device 1000 is made of a material including, but not limited to, metal, plastic, or combinations thereof, or when the syringe aspiration safety assistance device 1000 is made of an aluminum alloy, both are elastic materials used to construct the syringe aspiration safety assistance device 1000. Thus, when the thickness of the upper end of the plunger of a syringe U is greater than the distance between the syringe stop plate 2 and the syringe groove plate 3, the upper end of the plunger of the syringe U can pass through the syringe stop plate 2 and the syringe groove plate 3 made of an elastic material such that the syringe stop plate 2 and the syringe groove plate 3 forcibly clamp the upper end of the plunger of the syringe U through the elasticity of the material of the syringe stop plate 2 and the syringe groove plate 3, specifically through the elastic deformation of the syringe stop plate 2 and the syringe groove plate 3, thereby allowing the syringe stop plate 2 and the syringe groove plate 3 to be configured to receive the upper end of the plunger of the syringe U when the upper end of the plunger of the syringe U has variable thickness.

The edges of the syringe aspiration safety assistance device 1000 are subjected to rounded chamfer processing such that the syringe aspiration safety assistance device 1000 has a shape design that is more suitable for fitting to the thumb.

The syringe aspiration safety assistance device 1000 has a weight of 8 grams.

The reverse-buckle finger sleeve 1 has a one-way opening. The one-way opening ensures that the thumb of the operator has mobility in the reverse-buckle finger sleeve 1, thereby improving the stability during injection. Since the operator may not inject the entire contents of the syringe U at one time whenever the injection operation is performed, the operator often removes the syringe U, and the one-way opening ensures that the thumb of the operator has mobility and cannot be inadvertently touched or moved during removal of the syringe U to affect the syringe U that is half injected.

The end of the hook-shaped structure 11 of the reverse-buckle finger sleeve 1 is a trapezoidal structure. The trapezoidal structure enhances the convenience and mobility of the thumb entering and exiting the reverse-buckle finger sleeve 1, and prevents the operator's thumb entering and exiting the reverse-buckle finger sleeve 1 from inadvertently touching or moving the syringe U to affect the syringe U that is half injected.

In two preferred embodiments, the reverse-buckle finger sleeve 1 has two specifications to accommodate thumbs of different sizes, thereby allowing the thumb to remain mobile during use with the syringe aspiration safety assistance device 1000 of the disclosure.

Specification one—small-size reverse-buckle finger sleeve 1: when the length of the reverse-buckle finger sleeve 1 is 22 mm, the outer radius of the hook-shaped structure 11 is 9.5 mm. The small-size reverse-buckle finger sleeve 1 is suitable for thinner thumbs.

Specification two—large-size reverse-buckle finger sleeve 1: when the length of the reverse-buckle finger sleeve 1 is 24 mm, the outer radius of the hook-shaped structure 11 is 10.5 mm. The large-size reverse-buckle finger sleeve 1 is configured to accommodate the thumb thickness of a majority of adult users.

The shape of the syringe groove 31 of the syringe groove plate 3 includes but is not limited to a U-shape, which ensures that the syringe U does not slide during use, thereby improving operational stability.

An inner side of an upper end of the syringe groove plate 3 has a step.

The distance between the syringe stop plate 2 and the syringe groove plate 3 is 3 mm, so as to effectively correspond to and clamp the upper ends of most conventional syringe plungers.

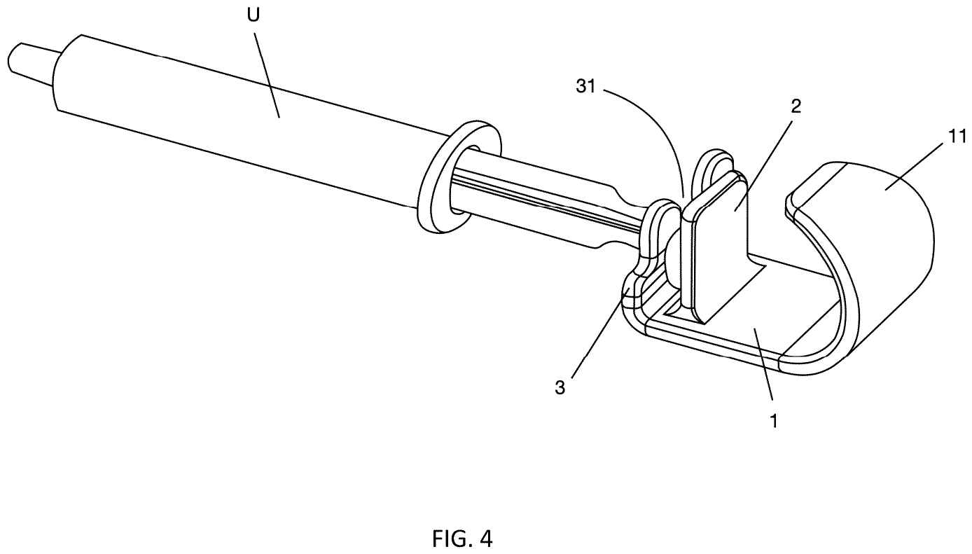

Referring to FIG. 4, there is shown a perspective view illustrative of the syringe aspiration safety assistance device fixing the syringe U in place according to the disclosure. As shown in the figure, after the upper end of the plunger of the syringe U has been secured between the syringe stop plate 2 and the syringe groove 31 of the syringe groove plate 3, a thumb of an injecting hand of an injector is inserted into the reverse-buckle finger sleeve 1 through the hook-shaped structure 11 formed by extending the upper end of the reverse-buckle finger sleeve 1, thereby permitting aspiration through a single-joint motion of the thumb of the injecting hand of the injector in a standard injection posture. After maintaining the aspiration action for several seconds, if the injector confirms that the needle tip of the syringe U is not in a blood vessel, the plunger can be pushed to perform the injection. After the injection is completed, the syringe aspiration safety assistance device 1000 of the present invention is removed from the syringe U, disinfected with alcohol, and can be reused for the next injection. Upon completion of the injection, the syringe aspiration safety assistance device 1000 of the disclosure is removed from the syringe U, disinfected with alcohol, and can be reused for the next injection.

The advantages of the syringe aspiration safety assistance device 1000 of the disclosure and the benefits of adopting the technology disclosed herein are as follows:
1. The syringe aspiration safety assistance device 1000 is compact and lightweight and thus does not affect the injection feel of the injector, thereby facilitating injection operations.
2. The syringe aspiration safety assistance device 1000 can be disinfected and reused.
3. The syringe aspiration safety assistance device 1000 is applicable to various forms of syringe U, and the process of fixing the syringe U in place is simple and fast, without increasing the injection time.
4. The syringe aspiration safety assistance device 1000 can, under a standard injection posture, perform aspiration using only a single-joint motion of the thumb of an injecting hand of the injector. This ensures high stability of the injection posture, greatly improves injection accuracy, and prevents displacement of the needle tip of the syringe U, thereby achieving consistency between the needle tip position during aspiration and during injection.
5. The reverse-buckle finger sleeve 1 of the syringe aspiration safety assistance device 1000 has a one-way opening, which is suitable for different thumb thicknesses of different injectors, and the hook-shaped structure 11 prevents the thumb from slipping, thereby solving the problem of lack of thumb mobility caused by the fully covering thumb designs of the prior art.
6. The syringe stop plate 2 and the syringe groove 31 of the syringe groove plate 3 of the syringe aspiration safety assistance device 1000 secure the upper end of the plunger of the syringe U and thereby directly fix the plunger of the syringe U in place, eliminating the need for an operator to continuously press the upper end of the plunger with a thumb during aspiration, and reducing the likelihood of device detachment during aspiration.
7. The syringe aspiration safety assistance device 1000 is made of a material including, but not limited to, metal, plastic, or combinations thereof, or the material is an aluminum alloy. Both are elastic materials for constructing the syringe aspiration safety assistance device 1000, and the elasticity of the material of the syringe aspiration safety assistance device 1000 enables the syringe aspiration safety assistance device 1000 to forcibly clamp the plunger of the syringe U, overcoming the limitations of non-elastic materials, which can accommodate only the original syringe U designs of major filler manufacturers currently on the market.

The syringe aspiration safety assistance device 1000 comprises a reverse-buckle finger sleeve 1, a syringe stop plate 2, and a syringe groove plate 3. The reverse-buckle finger sleeve 1 has an upper end that extends to form a hook-shaped structure 11, and a lower end connected to one end of the syringe stop plate 2. The syringe stop plate 2 has a lower end connected to an inner side of the syringe groove plate 3. The syringe groove plate 3 has a syringe groove 31. After the upper end of the plunger of the syringe U has been secured between the syringe stop plate 2 and the syringe groove 31 of the syringe groove plate 3, a thumb of an injecting hand of an injector is inserted into the reverse-buckle finger sleeve 1 through the hook-shaped structure 11 formed by extending the upper end of the reverse-buckle finger sleeve 1, thereby permitting aspiration through a single-joint motion of the thumb of the injecting hand of the injector in a standard injection posture, thereby improving stability, accuracy, and safety during aspiration.

Many alterations and modifications may be made by those having ordinary skill in the art without departing from the spirit and scope of the disclosed embodiments. Therefore, it must be understood that the illustrated embodiments have been set forth only for the purposes of example and that it should not be taken as limiting the embodiments as defined by the following claims. For example, even though the elements of a claim are set forth below in a certain combination, it must be expressly understood that the embodiment includes other combinations of fewer, more, or different elements disclosed herein even when not initially claimed in such combinations.

Thus, specific embodiments and applications of syringe aspiration safety assistance device have been disclosed. It should be apparent, however, to those skilled in the art that many more modifications besides those already described are possible without departing from the disclosed concepts herein. Therefore, the disclosed embodiments are not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Insubstantial changes from the claimed subject matter as viewed by a person with ordinary skill in the art, now known or later devised, are expressly contemplated as equivalent within the scope of the claims. Therefore, obvious substitutions now or later known to one with ordinary skill in the art are defined to be within the scope of the defined elements. The claims are thus to be understood to include what is specifically illustrated and described above, what is conceptually equivalent, what can be substituted, and what essentially incorporates the essential idea of the embodiments. In addition, where the specification and claims refer to at least one of something selected from the group consisting of A, B, C . . . and N, the text should be interpreted as requiring at least one element from the group which includes N, not A plus N, or B plus N, etc.

The words used in this specification to describe the various embodiments are to be understood not only in the sense of their commonly defined meanings but to include by special definition in this specification structure, material, or acts beyond the scope of the commonly defined meanings. Thus, if an element can be understood in the context of this specification as including more than one meaning, then its use in a claim must be understood as being generic to all possible meanings supported by the specification and by the word itself.

The definitions of the words or elements of the following claims therefore include not only the combination of elements which are literally set forth, but all equivalent structure, material or acts for performing substantially the same function in the same way to obtain the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

What is claimed is:

1. A syringe aspiration safety assistance device, comprising:
a syringe groove plate having a syringe groove;
a syringe stop plate having a lower end connected to an inner side of the syringe groove plate; and
a reverse-buckle finger sleeve having an upper end extending to form a hook-shaped structure, and a lower end connected to an end of the syringe stop plate.

2. The syringe aspiration safety assistance device of claim 1, wherein the syringe aspiration safety assistance device has a weight of 8 grams.

3. The syringe aspiration safety assistance device of claim 1, wherein the syringe aspiration safety assistance device is made of a material including, but not limited to, metal, plastic, or combinations thereof.

4. The syringe aspiration safety assistance device of claim 3, wherein the syringe stop plate and the syringe groove plate forcibly clamp an upper end of a plunger of a syringe through elasticity of the material of the syringe stop plate and the syringe groove plate.

5. The syringe aspiration safety assistance device of claim 4, wherein the syringe stop plate and the syringe groove plate undergo elastic deformation to receive the upper end of the plunger of the syringe when the upper end of the plunger of the syringe has variable thickness.

6. The syringe aspiration safety assistance device of claim 1, wherein the syringe aspiration safety assistance device is made of an aluminum alloy.

7. The syringe aspiration safety assistance device of claim 6, wherein the syringe stop plate and the syringe groove plate forcibly clamp an upper end of a plunger of a syringe through elasticity of the material of the syringe stop plate and the syringe groove plate.

8. The syringe aspiration safety assistance device of claim 7, wherein the syringe stop plate and the syringe groove plate undergo elastic deformation to receive the upper end of the plunger of the syringe when the upper end of the plunger of the syringe has variable thickness.

9. The syringe aspiration safety assistance device of claim 1, wherein edges of the syringe aspiration safety assistance device are subjected to rounded chamfer processing.

10. The syringe aspiration safety assistance device of claim 1, wherein the reverse-buckle finger sleeve has a one-way opening.

11. The syringe aspiration safety assistance device of claim 10, wherein the one-way opening and the hook-shaped structure are configured to allow movement of a thumb.

12. The syringe aspiration safety assistance device of claim 1, wherein an end of the hook-shaped structure of the reverse-buckle finger sleeve is a trapezoidal structure.

13. The syringe aspiration safety assistance device of claim 12, wherein the trapezoidal structure is configured to enhance convenience and mobility of a thumb entering and exiting the reverse-buckle finger sleeve.

14. The syringe aspiration safety assistance device of claim 1, wherein a length of the reverse-buckle finger sleeve is 22 mm, and an outer radius of the hook-shaped structure is 9.5 mm.

15. The syringe aspiration safety assistance device of claim 1, wherein a length of the reverse-buckle finger sleeve is 24 mm, and an outer radius of the hook-shaped structure is 10.5 mm.

16. The syringe aspiration safety assistance device of claim 1, wherein the syringe groove of the syringe groove plate has a shape including, but not limited to, a U-shape.

17. The syringe aspiration safety assistance device of claim 16, wherein the shape of the syringe groove ensures that a syringe clamped by the syringe stop plate and the syringe groove plate does not slide during use.

18. The syringe aspiration safety assistance device of claim 1, wherein an inner side of an upper end of the syringe groove plate has a step.

19. The syringe aspiration safety assistance device of claim 1, wherein a distance between the syringe stop plate and the syringe groove plate is 3 mm.

20. The syringe aspiration safety assistance device of claim 1, wherein aspiration is performed through a single-joint motion of a thumb in a standard injection posture.
